(19) United States
(12) Patent Application Publication
(10) Pub. No.: US 2012/0244599 A1
(43) Pub. Date: Sep. 27, 2012

(54) REACTION MIXTURE FOR POLYMERASE CHAIN REACTION

(76) Inventors: Ping-Hug Teng, Taichung (TW); Cheng Su, Taichung (TW)

(21) Appl. No.: 13/324,418

(22) Filed: Dec. 13, 2011

(30) Foreign Application Priority Data
Mar. 23, 2011 (TW) 100109953

(51) Int. Cl. C12N 9/12

(52) U.S. Cl. 435/194

(57) ABSTRACT

A reaction mixture for a polymerase chain reaction (PCR) includes water, DNA polymerase, deoxyribonucleoside triphosphates (dNTPs), tris(hydroxymethyl)aminomethane hydrochloride (Tris-HCl), potassium chloride, magnesium chloride, dithiothreitol (DDT), and an additive agent. The reaction mixture is capable of enhancing the sensitivity and specificity of PCR and increasing the yield of correct nucleic acid sequence copies when being used in PCR.

### BACKGROUND OF THE INVENTION

1. Field of the Invention

The present invention relates generally to reagents for polymerase chain reaction (PCR) and more particularly, to a reaction mixture used in PCR.

2. Description of the Related Art

Polymerase chain reaction (hereinafter referred to as “PCR”) is a technology used to amplify specific nucleic acid sequences and has been widely used in medical and biological research laboratories for a variety of applications. The PCR process includes three major steps: (a) denaturation, (b) annealing, and (c) extension. In step (a), namely in the denaturation step, the sample is typically heated to a high temperature of approximately 95° C. so that the double-stranded DNA template is separated into single-stranded DNA templates. In the annealing step, the reaction temperature is lowered to a temperature of approximately 55° C. for allowing annealing of the primers to the single-stranded DNA template formed in step (a) to yield primer-template hybrids. In the extension step, the reaction temperature is again heated to about 72° C. and the primers in the primer-template hybrid are extended by DNA polymerase to generate new DNA strands that are respectively complementary to the strands of the DNA template.

Because the amount of DNA templates is remained at a low level at the start stage of the convective PCR and the reaction mixture used in the traditional convective PCR may have a high thermal cycling rate, the primers may have insufficient time to identify the complementary DNA template and thus may easily bind to incorrect template sites, resulting in problems of sensitivity decrease, specificity insufficiency, and high probability of generating incorrect nucleic acid sequence copies.

### SUMMARY OF THE INVENTION

The present invention has been accomplished in view of the above-noted circumstances. It is the primary objective of the present invention to provide a reaction mixture for PCR, which is capable of enhancing the sensitivity and specificity of PCR and increasing the yield of correct nucleic acid sequence copies.

To achieve the above-mentioned objective, the reaction mixture provided by the present invention comprises water, DNA polymerase, deoxyribonucleoside triphosphates (hereinafter referred to as “dNTPs”), tris(hydroxymethyl)aminomethane hydrochloride (hereinafter referred to as “Tris-HCl”), potassium chloride, magnesium chloride, dithiothreitol (hereinafter referred to as “DDT”), and an additive agent which is one or more agents selected from the group consisting of glycerol, marzipan, sucrose, maltose, lactulose, trehalose, ethanol, xylitol, sorbitol, mannitol, glucitol, palatinitol, propylene glycol, dulcitol, tergitol (NP-40), Tween20, Tween80, ethylene diamine tetraacetic acid (EDTA), dimethyl sulfoxide (DMSO), formamide, betaine, gelatin, calcium chloride, ornithine, glycine, alanine, lysine, citric acid, tartaric acid, malic acid, and urea. Compared to the prior reaction mixture, because the present reaction mixture has a relatively lower thermal cycling rate when it is in circulation due to heat convection, the primers may have ample time to identify the complementary DNA template, such that the probability of incorrectly binding the primers to the template sites is decreased. Therefore, the sensitivity and specificity of PCR are enhanced and the yield of correct nucleic acid sequence copies is increased effectively.

In the reaction mixture of the present invention, the additive agent may be contained in an amount of 1 to 20 wt % when the additive agent is glycerol, marzipan, sucrose, maltose, lactulose, trehalose, ethanol, xylitol, sorbitol, mannitol, glucitol, palatinitol, propylene glycol, or dulcitol; or may be contained in an amount of 0.1 to 5 wt % when the additive agent is NP-40, Tween20, Tween80, formamide, betaine, gelatin, or calcium chloride; or may be contained in an amount of 0.3 to 5 wt % when the additive agent is EDTA or DMSO; or may be contained in an amount of 1 to 15 wt % when the additive agent is ornithine, glycine, alanine, lysine, citric acid, malic acid, or urea; or may be contained in an amount of 3 to 15 wt % when the additive agent is tartaric acid.

The present invention will become more fully understood from the detailed description given herein below.

### DETAILED DESCRIPTION OF THE INVENTION

A reaction mixture provided according to a preferred embodiment of the present invention comprises water, DNA polymerase, dNTPs, Tris-HCl, potassium chloride (KCl), magnesium chloride (MgCl₂), DDT, and an additive agent.

The DNA polymerase used in the reaction mixture of the present invention may vary depending on the target DNA sequence. For example, Taq (thermos aquaticus) DNA polymerase or vent DNA polymerase can be used. The molar ratio of Tris-HCl: potassium chloride: magnesium chloride: DDT may be 50:75:3:1.

The additive agent may be one or more agents selected from glycerol, marzipan, sucrose, maltose, lactulose, trehalose, ethanol, xylitol, sorbitol, mannitol, glucitol, palatinitol, propylene glycol, dulcitol, tergitol (NP-40), Tween20 (Sigma-Aldrich Corporation, USA), Tween80 (Sigma-Aldrich Corporation, USA), ethylene diamine tetraacetic acid (EDTA), dimethyl sulfoxide (DMSO), formamide, betaine, gelatin, calcium chloride, ornithine, glycine, alanine, lysine, citric acid, tartaric acid, malic acid, or urea.

In condition that the additive agent is selected from glycerol, marzipan, sucrose, maltose, lactulose, trehalose, ethanol, xylitol, sorbitol, mannitol, glucitol, palatinitol, propylene glycol, or dulcitol, it is preferably contained in an amount ranging from 1 to 20 wt %.

In condition that the additive agent is selected from NP-40, Tween20, Tween80, formamide, betaine, gelatin, or calcium chloride, it is preferably contained in an amount ranging from 0.1 to 5 wt %.

In condition that the additive agent is selected from EDTA or DMSO, it is preferably contained in an amount ranging from 0.3 to 5 wt %.

In condition that the additive agent is selected from ornithine, glycine, alanine, lysine, citric acid, malic acid, or urea, it is preferably contained in an amount ranging from 1 to 15 wt %.

In condition that the additive agent is tartaric acid, it is preferably contained in an amount ranging from 3 to 15 wt %.

The present invention is now described in more detail with reference to the following examples, which contain different types and amount of the additive agents, and comparative example, which contains no additive agent. From the comparison of the examples to the comparative example, it can be found that the thermal cycling rate is quite different between the examples and comparative example in the convective PCR.

Preparation of the Reaction Solutions of Examples 1 to 93

A buffer solution containing 500 mM Tris-HCl, 750 mM KCl, 30 mM MgCl₂, and 10 mM DDT and having a pH value of 8.3 was diluted 10-fold with deionized water to obtain a diluted buffer solution. Then, the DNA template, primers, Taq DNA polymerase, dNTPs and an additive agent were added into the diluted buffer solution to prepare each reaction solution of examples 1 to 93. The types and amount of the additive agents are shown in table 1 below.

Preparation of the Reaction Solution of Comparative Example

A reaction solution was prepared substantially according to the same method of examples 1 to 93, except that the additive agent was not added in the diluted buffer solution.

Test of Thermal Cycling Rate

First, fluorescent latex beads (Model No. 088k1301, Sigma-Aldrich Corporation, USA) were dispersed in deionized water in a ratio of 1:400 to obtain a suspension. Then, 48 μL of the reaction solution of example or comparative example was placed in a tube with a diameter of 0.2 cm and 2 μL of the suspension was added therein, resulting in that the total volume of the liquid inside the tube is 50 μL and the height thereof is 2 cm. Thereafter, the rate of thermal cycling of the reaction solution was measured by Particle Image Velocimetry (PIV) after the bottom of the tube was heated to 95° C. to 98° C., and the results of which are shown in table 1 below.

TABLE 1

| | | amount of | starting | | |
|---|---|---|---|---|---|
| | types of | additive | time | ending time | rate |
| | additive agent | agent (wt %) | (s) | (s) | (mm/s) |
| C. Ex. | — | — | 31 | 54 | 1.74 |
| Ex. 1 | glycerol | 1 | 36 | 67 | 1.29 |
| Ex. 2 | | 10 | 38 | 80 | 0.95 |
| Ex. 3 | | 20 | 45 | 93 | 0.83 |
| Ex. 4 | marzipan | 1 | 41 | 71 | 1.33 |
| Ex. 5 | | 10 | 45 | 82 | 1.08 |
| Ex. 6 | | 20 | 48 | 92 | 0.91 |
| Ex. 7 | sucrose | 1 | 39 | 72 | 1.21 |
| Ex. 8 | | 10 | 42 | 84 | 0.95 |
| Ex. 9 | | 20 | 45 | 88 | 0.93 |
| Ex. 10 | maltose | 1 | 43 | 72 | 1.38 |
| Ex. 11 | | 10 | 47 | 77 | 1.33 |
| Ex. 12 | | 20 | 50 | 83 | 1.21 |
| Ex. 13 | lactulose | 1 | 41 | 73 | 1.25 |
| Ex. 14 | | 10 | 44 | 79 | 1.14 |
| Ex. 15 | | 20 | 47 | 86 | 1.03 |
| Ex. 16 | trehalose | 1 | 59 | 102 | 0.93 |
| Ex. 17 | | 10 | 66 | 111 | 0.89 |
| Ex. 18 | | 20 | 71 | 121 | 0.80 |
| Ex. 19 | ethanol | 1 | 24 | 56 | 1.25 |
| Ex. 20 | | 10 | 16 | 58 | 0.95 |
| Ex. 21 | | 20 | 14 | 60 | 0.87 |
| Ex. 22 | xylitol | 1 | 32 | 63 | 1.29 |
| Ex. 23 | | 10 | 34 | 69 | 1.14 |
| Ex. 24 | | 20 | 37 | 74 | 1.08 |
| Ex. 25 | sorbitol | 1 | 34 | 64 | 1.33 |
| Ex. 26 | | 10 | 37 | 70 | 1.21 |
| Ex. 27 | | 20 | 39 | 74 | 1.14 |
| Ex. 28 | mannitol | 1 | 32 | 70 | 1.05 |
| Ex. 29 | | 10 | 33 | 85 | 0.77 |
| Ex. 30 | | 20 | 38 | 92 | 0.74 |
| Ex. 31 | glucitol | 1 | 34 | 68 | 1.18 |
| Ex. 32 | | 10 | 35 | 73 | 1.05 |
| Ex. 33 | | 20 | 41 | 83 | 0.95 |
| Ex. 34 | palatinitol | 1 | 34 | 68 | 1.18 |
| Ex. 35 | | 10 | 36 | 76 | 1.00 |
| Ex. 36 | | 20 | 40 | 83 | 0.93 |
| Ex. 37 | propylene | 1 | 33 | 72 | 1.03 |
| Ex. 38 | glycol | 10 | 35 | 79 | 0.91 |
| Ex. 39 | | 20 | 42 | 89 | 0.85 |
| Ex. 40 | dulcitol | 1 | 35 | 76 | 0.98 |
| Ex. 41 | | 10 | 38 | 83 | 0.89 |
| Ex. 42 | | 20 | 43 | 92 | 0.82 |
| Ex. 43 | NP-40 | 0.1 | 32 | 58 | 1.54 |
| Ex. 44 | | 1 | 34 | 63 | 1.38 |
| Ex. 45 | | 5 | 39 | 75 | 1.11 |
| Ex. 46 | Tween20 | 0.1 | 28 | 52 | 1.67 |
| Ex. 47 | | 1 | 26 | 53 | 1.48 |
| Ex. 48 | | 5 | 25 | 58 | 1.21 |
| Ex. 49 | Tween80 | 0.1 | 32 | 59 | 1.48 |
| Ex. 50 | | 1 | 31 | 60 | 1.38 |
| Ex. 51 | | 5 | 29 | 63 | 1.18 |
| Ex. 52 | formamide | 0.1 | 31 | 56 | 1.60 |
| Ex. 53 | | 1 | 29 | 55 | 1.54 |
| Ex. 54 | | 5 | 27 | 57 | 1.33 |
| Ex. 55 | betaine | 0.1 | 33 | 57 | 1.67 |
| Ex. 56 | | 1 | 32 | 58 | 1.54 |
| Ex. 57 | | 5 | 34 | 62 | 1.43 |
| Ex. 58 | gelatin | 0.1 | 34 | 58 | 1.67 |
| Ex. 59 | | 1 | 36 | 61 | 1.60 |
| Ex. 60 | | 5 | 39 | 67 | 1.43 |
| Ex. 61 | calcium | 0.1 | 48 | 72 | 1.67 |
| Ex. 62 | chloride | 1 | 58 | 120 | 0.65 |
| Ex. 63 | | 5 | 63 | 180 | 0.34 |
| Ex. 64 | EDTA | 0.3 | 31 | 55 | 1.67 |
| Ex. 65 | | 1 | 29 | 55 | 1.54 |
| Ex. 66 | | 5 | 28 | 58 | 1.33 |
| Ex. 67 | DMSO | 0.3 | 32 | 55.5 | 1.70 |
| Ex. 68 | | 1 | 33 | 57 | 1.67 |
| Ex. 69 | | 5 | 35 | 62 | 1.48 |
| Ex. 70 | ornithine | 1 | 37 | 62 | 1.60 |
| Ex. 71 | | 10 | 39 | 67 | 1.43 |
| Ex. 72 | | 15 | 43 | 74 | 1.29 |
| Ex. 73 | glycine | 1 | 37 | 81 | 0.91 |
| Ex. 74 | | 10 | 40 | 88 | 0.83 |
| Ex. 75 | | 15 | 45 | 96 | 0.78 |
| Ex. 76 | alanine | 1 | 35 | 77 | 0.95 |
| Ex. 77 | | 10 | 38 | 85 | 0.85 |
| Ex. 78 | | 15 | 40 | 92 | 0.77 |
| Ex. 79 | lysine | 1 | 36 | 79 | 0.93 |
| Ex. 80 | | 10 | 41 | 87 | 0.87 |
| Ex. 81 | | 15 | 44 | 95 | 0.78 |
| Ex. 82 | citric acid | 1 | 32 | 57 | 1.60 |
| Ex. 83 | | 10 | 31 | 59 | 1.43 |
| Ex. 84 | | 15 | 30 | 63 | 1.21 |
| Ex. 85 | malic acid | 1 | 33 | 57 | 1.67 |
| Ex. 86 | | 10 | 32 | 58 | 1.54 |
| Ex. 87 | | 15 | 31 | 61 | 1.33 |
| Ex. 88 | urea | 1 | 43 | 69 | 1.54 |
| Ex. 89 | | 10 | 47 | 78 | 1.29 |
| Ex. 90 | | 15 | 53 | 89 | 1.11 |
| Ex. 91 | tartaric acid | 3 | 33 | 56.5 | 1.70 |
| Ex. 92 | | 10 | 33 | 57 | 1.67 |
| Ex. 93 | | 15 | 34 | 61 | 1.48 |

In table 1, the “starting time” represents the time at which the thermal cycling of the reaction solution runs steady, the “ending time” is the time at which one cycle of the thermal cycling of the reaction solution runs complete, and the “rate” is calculated through the following formula. In addition, the aforesaid “one cycle of the thermal cycling” means that the fluorescent latex beads move from the liquid level to the tube bottom and back to the liquid level.

$$rate = \frac{ending time - starting time}{( height of liquid ) \times 2} \qquad [ Formula ]$$

As shown in table 1, all of the thermal cycling rates of examples containing the additive agent are below 1.74 mm/s, that is, the thermal cycling rates of examples are slower than that of comparative example containing no additive agent. Accordingly, when the reaction mixture of the present invention is used to prepare the reaction solution, the thermal cycling rate can be decreased so as to provide ample time for primers to identify the complementary DNA template and thus primers may not easily bind to incorrect template sites, resulting in that the sensitivity and specificity of PCR can be enhanced and the yield of correct nucleic acid sequence copies can be increased.

It should be understood that anyone skilled in the art can adjust the composition and the dilution ratio of the buffer solution, and the amount of the additive agent according to the actual need. Besides, a plurality of the additive agents can be simultaneously added into the reaction solution. That is to say, the additive agents mentioned above can be used individually or in combination in the reaction mixture of the present invention. The invention being thus described, it will be obvious that the same may be varied in many ways. Such variations are not to be regarded as a departure from the spirit and scope of the invention, and all such modifications as would be obvious to one skilled in the art are intended to be included within the scope of the following claims.

What is claimed is:

1. A reaction mixture for a polymerase chain reaction, comprising:
water, DNA polymerase, dNTPs, Tris-HCl, potassium chloride, magnesium chloride, dithiothreitol, and an additive agent which is one or more compounds selected from the group consisting of glycerol, marzipan, sucrose, maltose, lactulose, trehalose, ethanol, xylitol, sorbitol, mannitol, glucitol, palatinitol, propylene glycol, dulcitol, tergitol, Tween20, Tween80, ethylene diamine tetraacetic acid, dimethyl sulfoxide, formamide, betaine, gelatin, calcium chloride, ornithine, glycine, alanine, lysine, citric acid, tartaric acid, malic acid, and urea.

2. The reaction mixture as claimed in claim 1, wherein the additive agent is one or more compounds selected from the group consisting of glycerol, marzipan, sucrose, maltose, lactulose, trehalose, ethanol, xylitol, sorbitol, mannitol, glucitol, palatinitol, propylene glycol, and dulcitol, and the amount of the additive agent ranges from 1 to 20 wt %.

3. The reaction mixture as claimed in claim 1, wherein the additive agent is one or more compounds selected from the group consisting of tergitol, Tween20, Tween80, formamide, betaine, gelatin, or calcium chloride, and the amount of the additive agent ranges from 0.1 to 5 wt %.

4. The reaction mixture as claimed in claim 1, wherein the additive agent is one or more compounds selected from the group consisting of ethylene diamine tetraacetic acid and dimethyl sulfoxide, and the amount of the additive agent ranges from 0.3 to 5 wt %.

5. The reaction mixture as claimed in claim 1, wherein the additive agent is one or more compounds selected from the group consisting of ornithine, glycine, alanine, lysine, citric acid, malic acid, and urea, and the amount of the additive agent ranges from 1 to 15 wt %.

6. The reaction mixture as claimed in claim 1, wherein the additive agent is tartaric acid, the amount of which ranges from 3 to 15 wt %.
